(19) United States
(12) Patent Application Publication
(10) Pub. No.: US 2025/0177657 A1
(43) Pub. Date: Jun. 5, 2025

(54) Syringe Assembly with Automatic Safety Sleeve

(71) Applicant: Roncadelle Operations srl, Castel Mella (Bs) (IT)

(72) Inventor: Erik RYCKALTS, Castel Mella (Bs) (IT)

(21) Appl. No.: 18/839,963

(22) PCT Filed: Feb. 24, 2023
(86) PCT No.: PCT/EP2023/054700

§ 371 Date: Aug. 20, 2024

(30) Foreign Application Priority Data
Feb. 24, 2022 (EP) 22158585.4

(51) Int. Cl. A61M 5/32

(52) U.S. Cl. CPC A61M 5/3257

(57) ABSTRACT

The current invention relates to a syringe assembly comprising an outer barrel suitable to receive a drug compartment provided with a needle for injection and a stopper for sealing the proximal end of said drug compartment. The syringe assembly is further provided with a protective sleeve configured to be released by means of a button located at the distal end of the a plunger. Upon release, said protective sleeve is configured to extend through the outer barrel and over the whole length of the needle of the drug compartment. Once extended, the protective sleeve is retained is this extended position, rendering the syringe safe to handle.

### FIELD OF THE INVENTION

The present invention relates to medical and veterinarian devices. In particular, the invention relates to assemblies for injecting fluids.

### BACKGROUND

The use of a sharp-pointed piercing element entails the risk of accidental needle stick. The risk is particularly increased by the presence of potentially infectious biological material. To avoid such accidents, many prior art hypodermic syringes have included rigid cylindrical safety shields telescoped over the syringe barrel. These-shields can be moved between retracted positions where the needles are exposed for use, to extended positions where the needles are surrounded by the shields.

Such a device/composition according to the preamble is also known from WO 9014112. WO '112 describes a hypodermic syringe barrel provided with an external rotatable and slidable protective sleeve extendable and retractable relative to a needle mounted from the barrel and the sleeve is lightly biased toward the extended position thereof with the needle fully enclosed within the sleeve. a light tension spring causes the sleeve to be biased forward. This permits the user to slightly rotate the sleeve around the barrel, causing pin to be free to slide along the longitudinal slot and snap into a slot segment configured to prevent retraction of the sleeve when in the extended position.

EP 2018326161 describes a cylindrical safety device configured to fit over the distal end of a syringe, the device being spring-loaded and self-locking.

The described safety device comprises an outer tube within which the syringe barrel is slidably receivable. The outer tube has a distal end, a proximal end and a longitudinal axis. A collar is in the outer tube and is movable relative thereto. In some embodiments, the collar is fixedly attachable to the distal end of the syringe barrel. In other embodiments, the collar is rotatably attachable to the distal end of the syringe barrel. A force member, such as a compressible coil spring, is provided between the outer tube and the collar. The force member biases the outer tube in a distal direction. A track is formed in the inner surface of the outer tube. A pin extending radially outwardly from the collar slidingly engages said track which, in turn, guides the movement of the pin and therefore the collar within the outer tube.

Although the abovementioned documents disclose self-locking protective sleeves, the activation of said sleeves still requires a number of manipulation steps beyond the simple actuation of a plunger. Such additional manipulation steps require awkward one handed manipulation or, most likely, two handed manipulation, both which cases significantly increase the risk of accidental puncture.

U.S. Pat. No. 6,319,233 describes a safety shield system for a syringe, and such a system as used in combination with an assembly capable of functioning as a syringe. device is provided which includes an automatically operable shield system mounted to a syringe barrel. The system includes a holder which defines an enclosure. The syringe barrel extends at least partially, and preferably almost entirely, within the enclosure. The barrel is slidable within the holder. A retaining member is positioned on the holder, and is engageable with the barrel. This member prevents the barrel from being uncoupled from the holder. A shield is mounted to the holder, and positioned about at least a portion of the barrel. The shield is axially movable with respect to the holder between retracted and extended positions. It is intended to cover the needle tip when in the extended position. A spring engages the shield, and urges it towards the extended position. A first stop member is positioned on the shield, and a second stop member is positioned on the holder. The second stop member is engageable with the first stop member when the shield is in the retracted position. The force of the spring, by itself, is insufficient to cause disengagement of the first and second stop members. The barrel is operationally coupled to the shield such that sufficient axial movement of the barrel causes axial movement of the shield sufficient to cause disengagement of the first and second stop members. Such movement of the barrel is ordinarily caused by pressure exerted on the plunger rod by the user of the syringe following complete injection of the contents of the barrel.

The system disclosed in U.S. '233 permits the deployment of a protective element over the needle. However, the deployment of said protective element requires the pre-filled syringed to be movable inside the holder. The lack of firm attachment of the pre-filled syringe to the holder bear the risk of uncertainty regarding the positioning of the needle tip, therefore, the location where the drug is injected.

US20110046605A1 describes support device for a syringe, said support comprising a support sheath for the body of a syringe, and safety means which comprise an inner sheath. The inner sheet is slidable relative to said support sheath between a waiting retracted position, in which said inner sheath is substantially retracted inside said support sheath, and an active protection position, in which said inner sheath projects from said support sheath. The inner sheath has a free end portion formed by a flexible end-piece.

US20020193746A1 describes a safety support device for a syringe. The device comprises a support sheath for the body of the syringe and an inner sheath suitable for sliding relative to the support sheath between a waiting, retracted position and an extended position for protecting the needle of the syringe. The device has means for holding the syringe body relative to the support sheath and retaining means suitable for being activated to retain the inner sheath in the waiting, retracted position inside the support sheath and for being deactivated by the piston of the syringe to allow the inner sheath to extend into its protective, extended position.

The devices disclosed in U.S. '605 and U.S. '746 require the use of syringes equipped with a proprietary plunger rod in order to deploy their safety sheaths. This significantly reduces the compatibility of the devices with commonly available syringes.

The present invention aims to resolve at least some of the problems and disadvantages mentioned above. The aim of the invention is to provide a method which eliminates those disadvantages. The present invention targets at solving at least one of the aforementioned disadvantages.

The invention thereto aims to provide a syringe assembly that solves at least some of the problems mentioned above.

### SUMMARY OF THE INVENTION

The present invention and embodiments thereof serve to provide a solution to one or more of above-mentioned disadvantages. To this end, the present invention relates to syringe assembly according to claim 1 or claim 15.

The syringe assembly according to claim 1 comprises an outer barrel suitable to receive a drug compartment provided with a radially extending rim, a needle for injection for sealing the proximal end of said drug compartment. A protective sleeve configured to fit around said drug compartment and positioned inside the outer barrel provides a cover for the needle after injection. The protective sleeve has a first retracted position A and is biased by a spring towards a second extended position B. The protective sleeve is held at position A by means of a first set of at least two hooking means extending, which hooking means latch onto a first abutment near the distal end of the outer barrel. The syringe assembly according to claim 1 advantageously permits the deployment of the protective sleeve without any motions other than the displacement of the plunger in the distal direction.

Preferred embodiments of the device are shown in any of the claims 2 to 14. A second aspect of the invention is disclosed in claim 15. Claim 15 discloses a protective enclosure, which enclosure permits converting common drug compartments into safety syringes.

### DESCRIPTION OF FIGURES

The following description of the figures of specific embodiments of the invention is merely exemplary in nature and is not intended to limit the present teachings, their application or uses. Throughout the drawings, corresponding reference numerals indicate like or corresponding parts and features.

FIG. 1 shows a section view of the syringe assembly before administering the contents of the drug compartment.

FIG. 2 presents in more detail the proximal end of the syringe assembly with the plunger removed.

FIG. 3 presents an embodiment of a plunger equipped with a separate button.

FIG. 4 shows an embodiment of a plunger including an integral button.

FIG. 5 shows an embodiment of the syringe assembly after administration of most contents of the drug compartment.

FIG. 6 shows an embodiment of the syringe assembly after deployment of the protective sleeve.

### DETAILED DESCRIPTION OF THE INVENTION

The present invention concerns a syringe assembly. The syringe assembly according to the invention advantageously permits the deployment of a protective sleeve over an injection needle without any motions other than the displacement of the plunger in the distal direction. In this way unwanted contact with the needle after injection are advantageously prevented. Due to the reduced number of movements and force necessary to release the protective sleeve, the syringe assembly can easily be used with a single hand.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In a first aspect, the invention provides a syringe assembly comprising:
an outer barrel;
a drug compartment provided with a needle for injection and a stopper for sealing the proximal end of said drug compartment;
a protective sleeve at least partially fitted around said drug compartment and positioned inside the outer barrel and configured to cover the needle after injection;
a plunger present in the drug compartment; and
a spring configured to extend the protective sleeve in a distal direction and over the needle of the drug compartment;

The outer barrel has a first and a second internal abutment, wherein said first and second internal abutments are located at the proximity of the proximal end of said outer barrel, said second abutment has an internal perimeter which is smaller than an outer perimeter of a radially extending rim of the drug compartment. In this context, the terms at the proximity of the proximal end of said outer barrel is to be understood as near the proximal end of the outer barrel. In this invention, the first and second abutments extend radially from the internal surface of the outer barrel and towards the axis of said outer barrel. The protective sleeve has a first retracted position A and being biased by the spring towards a second extended position B, the protective sleeve being configured to be held at position A by means of a first set of at least two bendable hooking means located at the proximal end of the protective sleeve, which bendable hooking means are further configured to pass through passages located around the internal perimeter of the second abutment and to latch onto the first abutment. The syringe assembly according to the invention advantageously permits the deployment of the protective sleeve without any motions other than the displacement of the plunger in the distal direction. This advantageously permits the automatic deployment of said protective sleeve, which deployment does not leave the user in doubt regarding the safety of the syringe upon concluding the injection of the drug. By preference, the protective sleeve comes in contact with skin of the patient during the injection. In this way, a shock usually produced by the activations and extension of the protective sleeve is advantageously eliminated. The drug compartment with which the syringe assembly is equipped can be of many types, however, the invention is preferably applied to prefilled glass drug compartments according to ISO 11040 or similar.

In a preferred embodiment, the plunger has a button, said button being located at the proximal end of the plunger. This button permit and easier actuation of the bendable hooking means of protective sleeve. By preference, the button is attached to the plunger. In this way, common drug compartments can be made compatible with the syringe by attaching a button to the proximal end of drug compartment plunger, said button being capable of actuating the bendable hooking means of the protective sleeve. More preferably, the button is integral with the plunger. By preference, the plunger with integrated button is configured to be attached to the stopper of the drug compartment. In this way, the syringes can be distributed in advantageously more compact packages. More preferably, the one piece plunger and button may be provided already installed in the drug compartment. In this way, preparation and use of the syringe immediately before injection is made easier and faster, thereby further reducing the risk of injury by needle puncture.

In a further or another embodiment, the bendable hooking means have tips with beveled proximal outer edges, which beveled edges are further configured to be acted upon by the button. Said beveled edges permit directing a larger fraction of the force exerted by the button upon the bendable hooking means towards the longitudinal axis of the syringe assembly. This advantageously reduces the force that must be applied to the button in order to cause the bendable hooking means to disengage from the first abutment.

According to a further embodiment, the syringe assembly includes a protective sleeve bushing rigidly attached to the distal end of the outer barrel. This bushing permits the passage of the distal end of the protective sleeve. In this way, the bushing keeps the protective sleeve centered before and during deployment, thereby preventing any binding that might hinder the deployment of the protective sleeve. By preference, the protective sleeve comprises retaining means configured to retain said protective sleeve in position B. More preferably, the bushing includes retaining means, which retaining means are complimentary to the retaining means of the protective sleeve. The retaining means of the bushing are radially directed inwards towards the protective sleeve, said retaining means of the bushing having a beveled proximal surface. The retaining means of the protective sleeve being radially directed outwards and having a beveled distal surface suitable to override the retaining means of the bushing when the protective sleeve moves towards extended position B. When the protective sleeve is in position B, the proximal surface of the retaining means of the sleeve and the distal surface of the retaining means of the bushing oppose each other in order to prevent the sleeve from moving in a proximal direction. In this way, the protective sleeve is kept from retracting after it has been deployed, even when compressive force is applied on the distal end of the sleeve. This keeps the needle of the drug compartment permanently enshrouded, rendering the syringe assembly safe to handle after an injection. Another advantageous effect is that no additional features to be included in the drug compartment in order to retain the protective sleeve in advanced position B. This advantageously allows the use of common glass drug compartments. Yet another advantage associated, in particular, to the use of a bushing is that it permits easier production and assembly of the syringe.

In an embodiment, the bendable hooking means are bendable between an angle of −10° to 35°. Preferably, the bendable hooking means are bendable between an angle of −10° and 20°. More preferably, the bendable hooking means are bendable between an angle of −5° to 15°. This bending amplitude of the bendable hooking means advantageously allows for a positive engagement of the hooking means with the first abutment while permitting also easy and reliable disengagement of said hooking means from the second abutment when said hooking means are actuated by the distal end of the button.

According to an embodiment, the protective sleeve includes a larger diameter spring cavity near its proximal end and forward of the bendable hooking means. By preference, said spring cavity has a compression spring. More preferably still, the compression spring is positioned around the drug compartment. Most preferably, the proximal end of which compression spring rests against a distal face of the second abutment, and the distal end of said compression spring rests against the distal end of the spring cavity. The spring cavity and the second abutment provide an advantageous container for a compression spring as said cavity avoids any undesired deformation patterns of the coils of the spring, thereby ensuring a reliable transfer of tension to the protective sleeve.

In an embodiment, the means configured to retain the protective sleeve in advanced position B are a first hooking collar radially extending near the proximal end of the protective sleeve, and a second complimentary hooking collar directed inwards from the internal surface of the protective sleeve bushing. In this way, reliable hooking of the first hooking collar and the second hooking collar is advantageously guaranteed independently of the orientation of the protective sleeve relative to the bushing This advantageously permits using a simpler assembly process as the bushing does not need to be rotated to a specific orientation around its axis. By preference, the second hooking collar on the surface of the bushing is interrupted and located on the distal end of at least one bendable tab, said bendable tab being directed towards the distal end of the syringe. In this way, the force necessary to make the first hooking collar slide over the second complimentary hooking collar is advantageously reduced while maintaining a strong hooking action once the two hooking collars.

According to an embodiment the internal diameter of the first abutment is larger than the diameter of the radially extending rim of the drug compartment. This permits assembling and tensioning all elements of the syringe except the drug compartment and the button. In this way, the syringe assembly can be provided without a drug compartment installed. This advantageously permits delaying the choice of drug to be used with the device, thereby simplifying distribution and manufacturing cycle. In a preferred way the safety device is manufactured prior to inserting the drug compartment, whose drug compartment could be easily installed by drug manufacturers by just pushing said drug compartment into the device. In another preferred way of distributing the syringe, the syringe is provided with the drug compartments already installed.

The syringe assembly according to any of the previous claims, characterized in that, the outer barrel is further equipped with at least one inwards directed hooking tab at the proximal end of the outer barrel. By virtue of its beveled proximal surface, each of the at least one hooking tab is advantageously easy to be overridden by the radially extending rim of the drug compartment during the insertion of said drug compartment into the syringe. Once the drug compartment is fully inserted into the syringe, the at least one hooking tab prevents said drug compartment from falling off the syringe through its proximal end. By preference, said hooking tab is located closer to the proximal end of the outer barrel than the second abutment. More preferably, the distance between any said hooking tab and said abutment is at least equal to the thickness of the radially extending rim of the drug compartment. In this way, the drug compartment can simply be inserted through the proximal end of the syringe. Once inserted in the syringe, the drug compartment is safely retained between the hooking tab and the second abutment. By preference, the syringe has at least two hooking tabs, which hooking tabs occupy at least half of the inner perimeter near the proximal end of the outer barrel. More preferably, the at least two hooking tabs occupy at least 70% of the inner perimeter near the proximal end of the outer barrel. In this way, glass drug compartments having cut flanges as well as glass drug compartments having round flanges can be safely held inside the safety device. This coupling is possible just by pushing the glass compartment from the proximal toward the distal part of the safety device till the rim of said drug compartments are retained between the two hooking tabs and the second abutment. Furthermore, by having at least two hooking tabs occupy at least 70% of the inner perimeter near the proximal end of the outer barrel, rotation of the drug compartments during installation in the syringe is advantageously made unnecessary. This advantageously permits further simplification of the assembly process of the safety device with the glass drug compartment.

In a second aspect, the invention also relates to a protective enclosure suitable to receive a drug compartment, the enclosure comprising:
an outer barrel;
a protective sleeve;
a bushing attached to the distal end of the outer barrel; and
a spring configured to extend the protective sleeve in a distal direction through the distal end of the bushing;

The outer barrel has a first and a second internal abutment and at least one hooking tab, wherein said first and second internal abutments and hooking tabs are located at the proximity of the proximal end of said outer barrel, said second abutment has an internal perimeter which is smaller than an outer perimeter of a radially extending rim of the drug compartment. In an embodiment, the second abutment has an internal perimeter which is between 11 mm and 13 mm, more preferably between 11 mm and 12 mm. In this way, the enclosure can accommodate a range of drug compartments with a maximum volume of 0.5 ml up to a maximum volume of 3 ml. In an embodiment, the second abutment has an internal perimeter which is between 15 mm and 22 mm, more preferably the second abutment has an internal perimeter which is 22 mm. In this way, the enclosure can accommodate a range of drug compartments with a maximum volume of 0.5 ml up to a maximum volume of 20 ml. The protective sleeve included in the enclosure has a first retracted position A and being biased by the spring towards a second extended position B, the protective sleeve being configured to be held at position A by means of a first set of at least two bendable hooking means located at the proximal end of the protective sleeve, which bendable hooking means are further configured to pass through passages located around the internal perimeter of the second abutment and to latch onto the first abutment. The protective enclosure are preferably compatible with prefilled glass drug compartments according to ISO 11040 or similar. In this way, to convert an common drug compartment into a safety syringe, one must only drop said drug compartment into the enclosure. In this way, a manufacturer or user can procure drug compartments via at least a second supply chain. A main advantage of the enclosure is that, a manufacturer or user can convert an already existing stock of drug compartments into safety syringes. This permits, for example, health care practices, veterinary practices or the like to be brought into compliance with more stringent safety regulations without having to dispose of existing drug and drug compartment stock, thereby avoiding heavy costs.

It will be clear to a skilled person that above described embodiments of the syringe assembly can equally be applied to the protective enclosure.

The invention is further described by the following non-limiting examples which further illustrate the invention, and are not intended to, nor should they be interpreted to, limit the scope of the invention.

### DESCRIPTION OF FIGURES

With as a goal illustrating better the properties of the invention the following presents, as an example and limiting in no way other potential applications, a description of a number of preferred embodiments of the safety syringe, wherein:

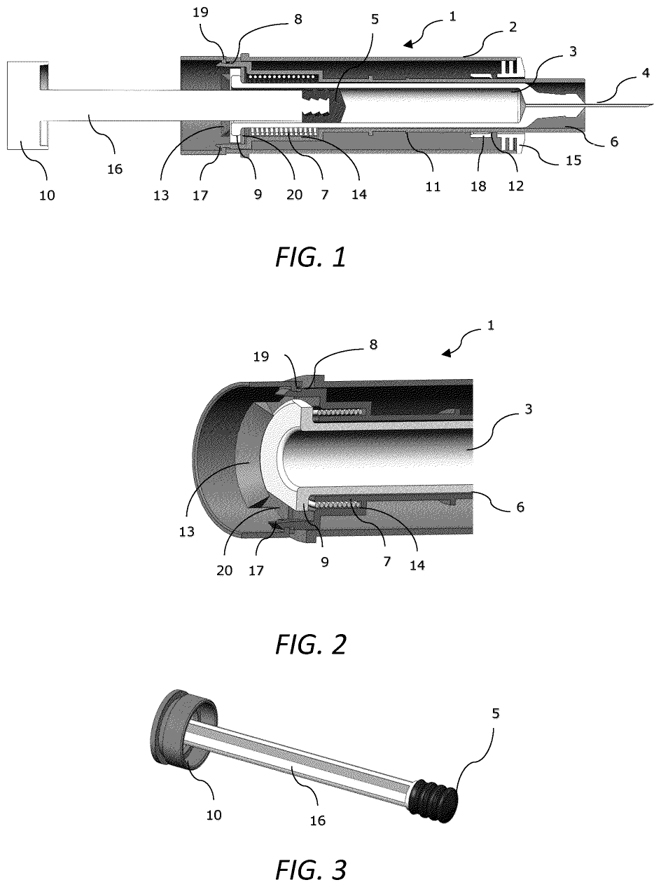

FIG. 1 shows a section view of the syringe assembly 1 before administering the contents of the drug compartment 3. In this figure, a drug compartment 3 is shown installed inside an outer barrel 2. Said drug compartment 3 comprises a needle 4, a stopper 5 and a radially extending rim 9, which rim 9 is retained between a second abutment 20 and a hooking tab 13, thereby retaining the drug compartment 3 immobilized inside the outer barrel 2. A protective sleeve 6 is shown around the drug compartment 3 and extending through a bushing 15 and towards the distal end of the syringe assembly 1. A set of bendable hooking means 8 with beveled tips 17 is shown latched onto a first abutment 19. A spring chamber 14 is shown containing a spring 7 compressed between the second abutment 20 and the distal end of said spring chamber 14. A plunger 16 with an integral button 10 is shown rigidly attached to the stopper 5. The protective sleeve is shown further equipped with a first hooking collar 11, said hooking collar 11 being configured to latch onto the second hooking collar 12 formed by the distal end of the bendable tab 18 near the proximal end of the bushing 15.

FIG. 2 presents in more detail the proximal end of the syringe assembly 1 with the plunger 16 removed. In this figure, the protective sleeve 6 is presented in position A, the bendable hooking means 8 being shown latching onto the first abutment 19. The drug compartment 3 is shown attached to the outer barrel 2 by means of its rim 9, which rim 9 is shown caught between the second abutment 20 and the hooking tab 13. The spring 7 is still shown compressed in its cavity 14.

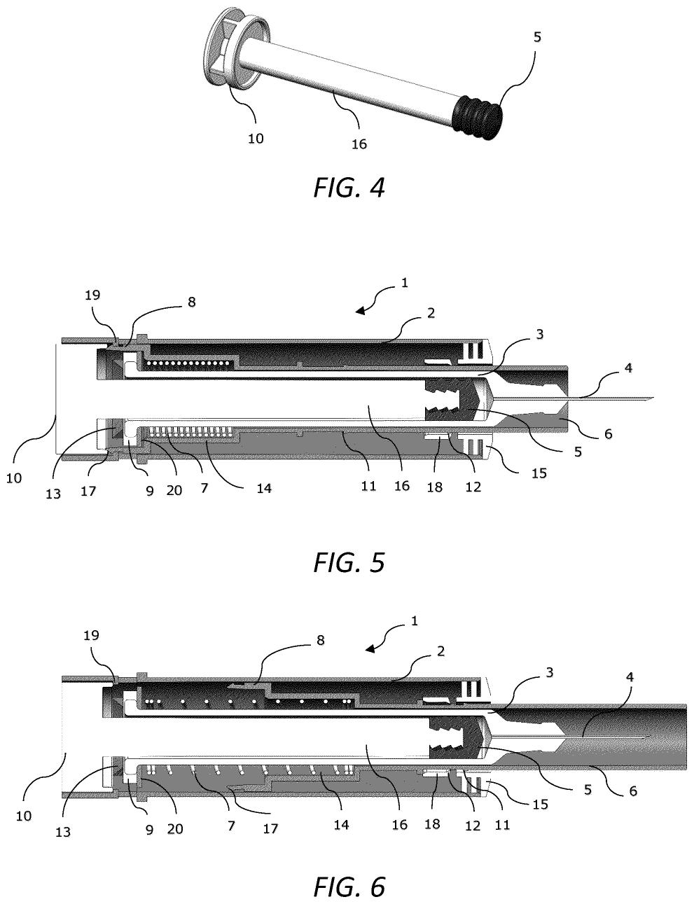

FIG. 3 presents an embodiment of a plunger 16 equipped with a separate button 10. The button 10 is shown attached to the proximal end of the plunger 16, giving said plunger 16, the same functional characteristics of the plunger 16 as represented in FIG. 4, which figure shows an embodiment of a plunger 16 including an integral button 10.

FIG. 5 shows an embodiment of the syringe assembly 1 after administration of most contents of the drug compartment 3. In this figure, the plunger 16 and the stopper 5 have been pushed inside the drug compartment 3 almost completely. The figure shows how, at this stage, the distal end of the button 10 rests against the bevel of the tips of the bendable hooking means 17. All other elements of the syringe are shown in the same position as in FIG. 1. FIG. 6 shows the next stage in the operation of the syringe 1.

FIG. 6 shows an embodiment of the syringe assembly 1 after deployment of the protective sleeve 6. The figure shows the plunger 16 and the stopper 5 pushed further into their final position inside the drug compartment 3, with the button 10 now resting against first abutment 17. The protective sleeve 6 is now shown in an advanced position B, having traveled in the distal direction under action of spring 7.

The sleeve 6 is now shown extending past the bushing 15 and completely covering the needle 4 while the drug compartment 3 is still retained by its rim 9, which rim 9 is still between the second abutment 20 and the hooking tab 19. The first hooking collar 11 is shown past the second hooking collar 12, said first hooking collar 11 having overcome the tension of the bendable tab 18 and in this way locking the protective sleeve 6 in advanced position B.

### LIST OF NUMBERED ITEMS

1 syringe assembly
2 outer barrel
3 drug compartment
4 needle
5 stopper
6 protective sleeve
7 spring
8 bendable hooking means
9 radially extending rim
10 button
11 first hooking collar
12 second hooking collar
13 hooking tab
14 spring cavity
15 bushing
16 plunger
17 tip of bendable hooking means
18 bendable tab
19 first abutment
20 second abutment

1. A syringe assembly comprising:
an outer barrel;
a drug compartment provided with a needle for injection and a stopper for sealing a proximal end of said drug compartment;
a protective sleeve at least partially fitted around said drug compartment and positioned inside the outer barrel and configured to cover the needle after injection;
a plunger present in the drug compartment; and
a spring configured to extend the protective sleeve in a distal direction and over the needle of the drug compartment;
characterized in that, the outer barrel has a first and a second internal abutment, wherein said first and second internal abutments are located at a proximity of a proximal end of said outer barrel, said second abutment has an internal perimeter which is smaller than an outer perimeter of a radially extending rim of the drug compartment; the syringe assembly, further characterized in that, the protective sleeve has a first retracted position A and being biased by the spring towards a second extended position B, the protective sleeve being configured to be held at the position A by means of a first set of at least two bendable hooking means located at a proximal end of the protective sleeve, wherein the bendable hooking means are further configured to pass through passages located around the internal perimeter of the second abutment and to latch onto the first abutment.

2. The syringe assembly according to claim 1, characterized in that, the plunger has a button, said button being located at a proximal end of the plunger.

3. The syringe assembly according to claim 1, characterized in that, the bendable hooking means have tips with beveled proximal outer edges, wherein the beveled proximal outer edges are further configured to be acted upon by the button.

4. The syringe assembly according to claim 1, characterized in that, the syringe assembly includes a bushing rigidly attached to a distal end of the outer barrel.

5. The syringe assembly according to claim 1, characterized in that, the protective sleeve comprises retaining means configured to retain said protective sleeve in the position B.

6. The syringe assembly according to claim 4, characterized in that, the bushing includes retaining means, wherein the retaining means are complimentary to the retaining means of the protective sleeve.

7. The syringe assembly according to claim 1, characterized in that, the bendable hooking means are bendable between an angle of −10° to 35°.

8. The syringe assembly according to claim 1, characterized in that, the protective sleeve includes a larger diameter spring cavity near the proximal end of the protective sleeve and forward of the bendable hooking means.

9. The syringe assembly according to claim 8, characterized in that, the spring cavity has a compression spring.

10. The syringe assembly according to claim 9, characterized in that, the compression spring is positioned around the drug compartment.

11. The syringe assembly according to claim 5, characterized in that, the retaining means configured to retain the protective sleeve in the position B are a first hooking collar radially extending near the proximal end of the protective sleeve, and a second complimentary hooking collar directed inwards from an internal surface of the bushing.

12. The syringe assembly according to claim 1, characterized in that, an internal diameter of the first abutment is larger than a diameter of the radially extending rim of the drug compartment.

13. The syringe assembly according to claim 1, characterized in that, the outer barrel includes at least one inwards directed hooking tab at the proximal end of the outer barrel.

14. The syringe assembly according to claim 13, characterized in that, the hooking tab is located closer to the proximal end of the outer barrel than the second abutment.

15. A protective enclosure suitable to receive a drug compartment, the enclosure comprising:
an outer barrel;
a protective sleeve positioned inside the outer barrel;
a bushing rigidly attached to a distal end of the outer barrel; and
a spring configured to extend the protective sleeve in a distal direction through a distal end of the bushing;
characterized in that, the outer barrel has a first and a second internal abutment and at least one hooking tab, wherein said first and second internal abutments and the hooking tabs are located at a proximity of a proximal end of said outer barrel, said second abutment has an internal perimeter which is smaller than an outer perimeter of a radially extending rim of the drug compartment;
the enclosure, further characterized in that, the protective sleeve has a first retracted position A and being biased by the spring towards a second extended position B, the protective sleeve being configured to be held at position A by means of a first set of at least two bendable hooking means located at the proximal end of the protective sleeve, wherein the bendable hooking means are further configured to pass through passages located around the internal perimeter of the second abutment and to latch onto the first abutment.
